(19) United States
(12) Patent Application Publication
(10) Pub. No.: US 2025/0222462 A1
(43) Pub. Date: Jul. 10, 2025

(54) ELECTRODE SYSTEM AND CELL RECORDING DEVICE INCLUDING THE SAME

(71) Applicant: Samsung Electronics Co., Ltd., Suwon-si (KR)

(72) Inventors: Kitae PARK, Suwon-si (KR); Chisung BAE, Suwon-si (KR)

(73) Assignee: Samsung Electronics Co., Ltd., Suwon-si (KR)

(21) Appl. No.: 18/946,717

(22) Filed: Nov. 13, 2024

(30) Foreign Application Priority Data
Jan. 10, 2024 (KR) 10-2024-0004381

(51) Int. Cl. B01L 9/00; C12M 1/00; C12M 1/32

(52) U.S. Cl. CPC B01L 9/00; C12M 23/12; C12M 23/30; B01L 2300/0645

(57) ABSTRACT

An electrode system and a cell recording device including the same are provided. The electrode system includes a multi-electrode array (MEA) layer, and a well array layer disposed to support a cell culture and disposed on the MEA layer, and wherein the well array layer further includes a degradable material.

### CROSS-REFERENCE TO RELATED APPLICATIONS

This application claims the benefit under 35 USC § 119(a) of Korean Patent Application No. 10-2024-0004381 filed on Jan. 10, 2024, in the Korean Intellectual Property Office, the entire disclosure of which is incorporated herein by reference for all purposes.

### BACKGROUND

### 1. Field

The following description relates to an electrode system, and a cell recording device including the same.

### 2. Description of Related Art

Technologies that implement various types of neural networks in a test tube to observe dynamic communication between neurons, or activation functions of a typical neural network, continue to be developed. With the development of three-dimensional (3D) culture technology, an interest in a modular nerve cell network is growing. Most existing neural networks implemented in vitro have an all-to-all random network structure.

To culture nerve cells in 3D, instead of on a plane, a floating culture may be performed to allow cells to naturally aggregate together to form a network (e.g., spheroids), or a network may be formed in 3D in a stereoscopic structure, such as a scaffold, and the like.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In a general aspect, an electrode system includes a multi-electrode array (MEA) layer; and a well array layer, disposed to support a cell culture, and disposed on the MEA layer, wherein the well array layer comprises a degradable material.

A micro-sized well may be disposed in the well array layer, a bottom surface of the well may have at least one of a flat shape, a rounded shape, and a conical shape, and the well may have a width that decreases in a downwards direction or has a predetermined width.

A well that is disposed in the well array layer may have a width of 1 micrometer (μm) to 500 μm.

A well that is disposed in the well array layer may have a maximum width of 100 μm to 1,000 μm, and a minimum width of 1 μm to about 1,000 μm.

The well array layer may include a degradable polymer, a degradable hydrogel, or both, and each of the degradable polymer and the degradable hydrogel may have a form of one of a fiber and a particle, or may be formed as a cross-linked network.

The well array layer may be degraded and removed by one of a mechanical factor, a physical factor, and a chemical factor.

The well array layer may be degraded by a factor including at least one of a temperature, a light, a change in pH, a vibration, an ultrasonic wave, and an acid.

The MEA layer may include a plurality of electrode units, a first portion of the plurality of electrode units is arranged in an area corresponding to an area of wells of the well array layer, and a second portion of the plurality of electrode units are arranged between the wells of the well array layer, and each of the electrode units may include a single electrode or a plurality of electrodes.

An electrode of the MEA layer may be surface-modified or surface-textured with a chemical functional group.

The electrode system may include an adhesion layer disposed between the well array layer and the MEA layer.

The adhesion layer may have a thickness of 0.01 μm to 1 μm.

Cells may be floating-cultured into spheroids within a well that is disposed in the well array layer, the spheroids may be in contact with an electrode layer corresponding to the well and are adherent-cultured, after the well is removed, and the spheroids may form a connection network with each other during the adherent culturing.

The cells may be nerve cells, and the connection network may be formed between the spheroids through a growth of axons during the adherent culturing.

The electrode system may be configured to measure cell information during or after the adherent culturing of the spheroids.

The electrode system may be configured to measure an electrical signal of the nerve cells.

In a general aspect, a cell recording device includes an electrode system including a multi-electrode array (MEA) layer; and a well array layer, disposed to support a cell culture, and disposed on the MEA layer, wherein the well array layer includes a degradable material.

The cell recording device may be configured to culture cells in vitro or in vivo, collect cell information, stimulate cells, or electroporate cells.

The cell recording device may be an electrophysiological system-on-a-chip (SOC) to record a signal of a nerve cell or stimulate a nerve cell.

The degradable material may include one of a degradable polymer and a degradable hydrogel.

Micro-sized wells may be patterned on the MEA, and a spheroid network may be patterned on a surface of the MEA.

Other features and aspects will be apparent from the following detailed description, the drawings, and the claims.

### BRIEF DESCRIPTION OF DRAWINGS

FIG. 1 illustrates an example configuration of an example electrode system, in accordance with one or more embodiments.

FIG. 2 illustrates another example configuration of an example electrode system, in accordance with one or more embodiments.

FIG. 3 illustrates another example configuration of an example electrode system, in accordance with one or more embodiments.

FIG. 4 illustrates another example configuration of an example electrode system, in accordance with one or more embodiments.

FIG. 5 illustrates another example configuration of an example electrode system, in accordance with one or more embodiments.

FIG. 6 illustrates an example integration process of a connection network of spheroids and a multi-electrode array (MEA) layer of an example electrode system, in accordance with one or more embodiments.

FIG. 7 illustrates another example integration process of a connection network of spheroids and an MEA layer of an example electrode system, in accordance with one or more embodiments.

FIG. 8 illustrates an example arrangement pattern of a connection network of spheroids and an MEA layer of an example electrode system, in accordance with one or more embodiments.

FIG. 9 is a flowchart illustrating an example method of forming a modular network of an example electrode system, in accordance with one or more embodiments.

FIG. 10 is a flowchart illustrating an example method of recording cells using an electrode system, in accordance with one or more embodiments.

Throughout the drawings and the detailed description, unless otherwise described or provided, it may be understood that the same drawing reference numerals may refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

The features described herein may be embodied in different forms and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application. The use of the term “may” herein with respect to an example or embodiment, e.g., as to what an example or embodiment may include or implement, means that at least one example or embodiment exists where such a feature is included or implemented, while all examples are not limited thereto. The use of the terms “example” or “embodiment” herein have a same meaning, e.g., the phrasing “in one example” has a same meaning as “in one embodiment”, and “one or more examples” has a same meaning as “in one or more embodiments.”

The terminology used herein is for describing various examples only and is not to be used to limit the disclosure. The articles “a,” “an,” and “the” are intended to include the plural forms as well, unless the context clearly indicates otherwise. As used herein, the term “and/or” includes any one and any combination of any two or more of the associated listed items. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

Unless otherwise defined, all terms including technical and scientific terms used herein have the same meaning as commonly understood by one of ordinary skill in the art to which examples belong. Terms, such as those defined in commonly used dictionaries, are to be interpreted as having a meaning that is consistent with their meaning in the context of the relevant art and the present disclosure, and are not to be interpreted in an idealized or overly formal sense unless expressly so defined herein.

Throughout the specification, when a component or element is described as being “on”, “connected to,” “coupled to,” or “joined to” another component, element, or layer it may be directly (e.g., in contact with the other component, element, or layer) “on”, “connected to,” “coupled to,” or “joined to” the other component, element, or layer or there may reasonably be one or more other components, elements, layers intervening therebetween. When a component, element, or layer is described as being “directly on”, “directly connected to,” “directly coupled to,” or “directly joined” to another component, element, or layer there can be no other components, elements, or layers intervening therebetween. Likewise, expressions, for example, “between” and “immediately between” and “adjacent to” and “immediately adjacent to” may also be construed as described in the foregoing.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

As used herein, the term “and/or” includes any one and any combination of any two or more of the associated listed items. The phrases “at least one of A, B, and C”, “at least one of A, B, or C”, and the like are intended to have disjunctive meanings, and these phrases “at least one of A, B, and C”, “at least one of A, B, or C”, and the like also include examples where there may be one or more of each of A, B, and/or C (e.g., any combination of one or more of each of A, B, and C), unless the corresponding description and embodiment necessitates such listings (e.g., “at least one of A, B, and C”) to be interpreted to have a conjunctive meaning.

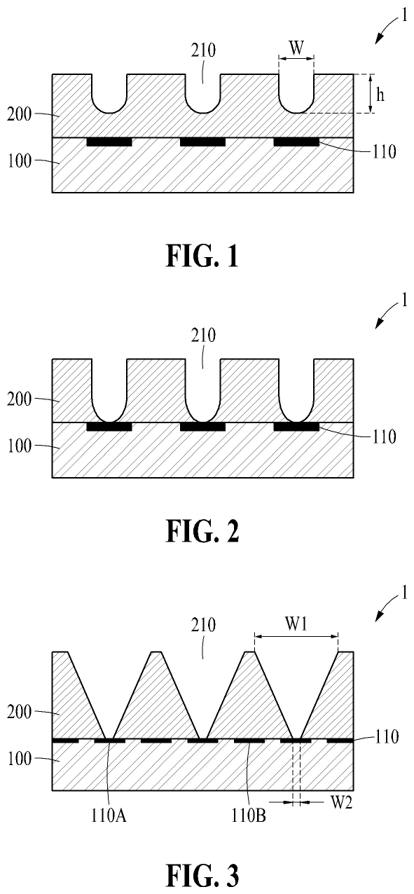

FIGS. 1, 2, and 3 illustrate examples of a configuration of an example electrode system 1.

Referring to FIG. 1, the electrode system 1 may include a multi-electrode array (MEA) layer 100, and a well array layer 200 disposed to support a cell culture, and disposed on the MEA layer 100.

In an example, the MEA layer 100 may be patterned to include a plurality of electrode units 110. In an example, the electrode units 110 may each include a single electrode or a plurality of electrodes. In another example, the electrode units 110 may be patterned to be regularly arranged or disposed.

In an example, the MEA layer 100 may be at least partially inserted in a direction of a depth of a support substrate, and may include a planar electrode with an exposed upper area and a protruding electrode with a protruding upper area.

In an example, an electrode (e.g., an electrode unit 110) may have a shape of a line, a circle, a dot, an oval, or a polygon, as only examples. For example, the MEA layer 100 may be a micro electrode array.

In an example, the MEA layer 100 may include a material that provides an electrode characteristic. In an example, an electrode may include, as only examples, a metal, a metal oxide, an alloy, a carbon-based material, or a conductive polymer.

In an example, an electrode (e.g., an electrode unit 110) of the MEA layer 100 may be surface-modified. In an example, due to a surface modification, an adhesive force that is added to cells (e.g., spheroids) may be enhanced. In another example, the surface modification may be performed by a hydrophilic functional group (e.g. a thiol group, a hydroxyl group, and a carboxyl group) or a lipophilic functional group. In another example, due to the surface modification, a surface roughness may increase, or a surface texture may be formed. In an example, the surface texture may be a nano-texture or a micro-texture, and may include, for example, an embossed texture, a molded texture, a patterned texture, or an etched surface texture. In another example, the surface texture may be a regular or irregular arrangement.

In an example, at least a portion of the plurality of electrode units 110 may be arranged in an area corresponding to a well 210, as illustrated in FIG. 1. The area corresponding to the well 210 may be an area corresponding to the well 210 that is disposed vertically with respect to the well array layer 200 and the MEA 100. In an example, a portion of the plurality of electrode units 110 may be arranged in an area corresponding to the well 210. In an example, a portion of the electrode units 110 may be disposed in an area corresponding to the well 210, and the remaining electrode units 110 may be arranged or disposed between the wells 210, as illustrated in FIG. 3. In an example, after a floating culture, cell spheroids may be in contact with an electrode 110A, and a network connection portion between spheroids may be in contact with an electrode 110B.

According to an example, the MEA layer 100 may be a single layer, or a layer formed by laminating a plurality of layers. For example, the plurality of layers may independently perform functions, and a measurement of cell information, electroporation of cells, or cell stimulation may be sequentially or simultaneously performed.

In an example, a floating culture, as a cell culture, may be performed on the well array layer 200, and the well array layer 200 may include a material that has a low adhesion rate of cells on an inner surface (e.g., a side surface and a bottom surface) of a well 210 or to which cells hardly adhere. In an example, the well array layer 200, which is a degradable well, may include a degradable material. In an example, the degradable material may include a degradable polymer or a degradable hydrogel. In an example, the term “degradable” may be defined as capable of being self-degradable or degradable by an external factor (or an external environment). For example, external factors may be used to control a degradation time or a degradation rate. In another example, the term “degradable” may be defined as capable of being degraded or removed by a physical factor, a chemical factor, or a mechanical factor. For example, the term “factor” may be a temperature (e.g., heating), light, a vibration, an ultrasonic wave, a rotation (e.g., a centrifugal force), a change in pH, an acid, a base, an enzyme, a hydrolysis agent, and the like.

According to an example, the degradable polymer or the degradable hydrogel may include, for example, a biopolymer material or hydrogel that has a low toxicity to cells or a human body and that is capable of being degraded by the above-described factors, but is not limited thereto. In an example, the degradable polymer or the degradable hydrogel may include one or at least two selected from naturally-derived degradable alginate, agarose, chitosan, collagen, hyaluronic acid, fibrin, methylcellulose, peptide, polylactic acid, polycaprolactone, poly(hydroxybutyrate), poly(orthoester), poly(α-hydroxyester), polyethylene oxide (PEO), polypropylene oxide (PPO), or a copolymer thereof. The above copolymer may be a binary copolymer or a terpolymer.

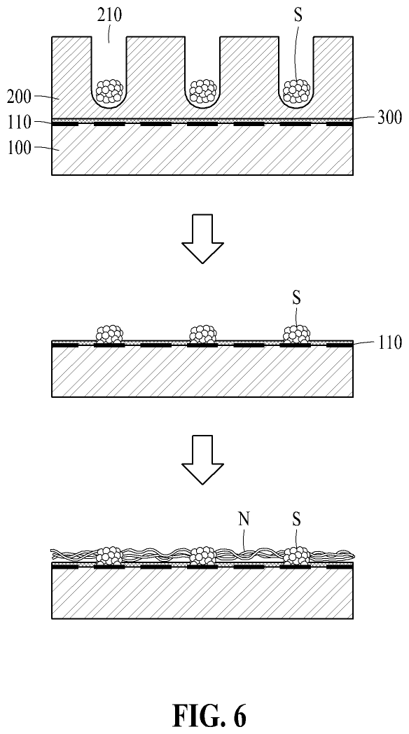

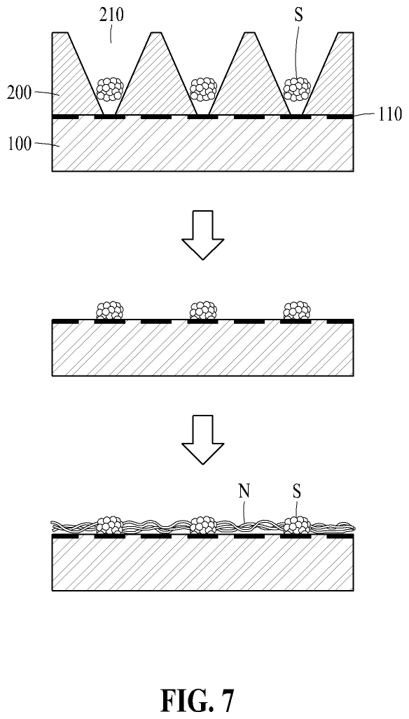

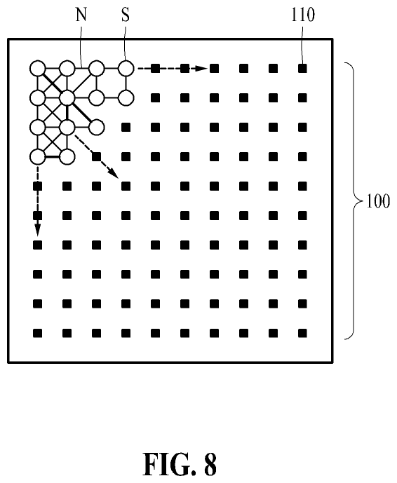

In an example, FIGS. 6 and 7 illustrate examples of an integration process of a connection network of spheroids and an MEA layer 100 of an electrode system. FIG. 8 illustrates an example arrangement pattern of a connection network of spheroids and an MEA layer 100 of an electrode system.

Referring to FIGS. 6 to 8, a well array layer 200 may culture (e.g., floating culture) cells into spheroids S, and may control an adhesion position or an adhesion strength between the cultured spheroids and a surface of the MEA layer 100. In an example, cells may aggregate in the well 210 and may be grown into spheroids or organoids. In an example, the well array layer 200 may include a patterned well by implementing the above adhesion position, as illustrated in FIG. 8. In an example, the well array layer 200 may set a degradation time or a degradation rate and control a time at which a spheroid S and the MEA layer 100 contact or a time at which a connection network N of spheroids S is formed. In an example, the well array layer 200 may be removed when culturing of spheroids or organoids is completed.

In an example, a degradable material (e.g., a degradable polymer or a degradable hydrogel) may be included in the form of a particle, a fiber, a wire, a needle, a load or a sheet, or may be included as a cross-linked network in the well array layer 200. In an example, particles may have spherical, polygonal or elliptical shapes, as only examples. In an example, the degradable material may be nano-sized or greater, or micro-sized. In an example, the degradable material may be included in a volume corresponding to 10% or greater; 30% or greater; 50% or greater; 70% or greater; 80% or greater; 90% or greater; 95% or greater; or 100% of the total volume in a well layer. Within the above-described volume range, a maximum value, a minimum value, or an average value may be selected.

In an example, the well array layer 200 may induce an adherent culture after a floating culture through a degradation or removal process, or may control an adherent culture time or an adherent culture position, which may be implemented through an adjustment of a degradation rate or a degradation time of the well array layer 200. The well array layer 200 may be patterned based on an arrangement (or a pattern) of the MEA layer 100 and may implement a position in which a spheroid is attached and cultured (e.g., adherent culture). In an example, the well array layer 200 may perform culturing, as a floating culture, in the form of spheroids or organoids within a well.

In an example, referring to FIGS. 6 and 7, a floating culture of cells may be performed in the well array layer 200 to form spheroids S, and the spheroids S may be in contact with the MEA layer 100 when the well array layer 200 is degraded and removed. An adherent culture of the spheroids S may be performed on the MEA layer 100 to form a network between the spheroids S. The network may extend through an electrode layer between spheroids S, and information generated in spheroids S and a network of the spheroids S may be obtained.

In an example, referring to FIG. 8, spheroids S may form connection networks N according to an arrangement (or a pattern) of electrode units 110. Accordingly, patterned connection networks N may be formed, and an in-situ integration of electrodes, spheroids, and a modularized network thereof may be implemented. In FIG. 8, the connection networks N may be extended or expanded as, for example, indicated by arrows, to be formed on at least a portion of the MEA layer 100 or the entire surface of the MEA layer 100.

In an example, cells and spheroids may be nerve cells or spheroids of nerve cells, and networks thereof may be formed by an extension or a growth of projections (e.g., axons) of nerve cells. The above networks may form a modular neural network between spheroids coupled to an electrode unit 110.

In an example, the well 210 may be micro-sized and may have a height (e.g., a height “h” of FIG. 1) of about, as examples, 10 micrometers (μm) to about 1,000 μm, and a diameter (or a width) (e.g., a width “W” of FIG. 1) of about, as examples, 1 μm to about 1,000 μm; or about 1 μm to about 500 μm. The above-described values may be a maximum value, a minimum value, or an average value. In an example, a diameter (or a width) in the well 210 may remain unchanged, or may decrease or increase downwards (e.g., towards the MEA layer 100) according to a shape of a well (e.g., FIGS. 1 and 3). In an example, at least one well 210 in the well array layer 200 may have a maximum width (e.g., “W1” of FIG. 3) of about, as examples, 100 μm to about 1,000 μm; about 200 μm to about 900 μm; or about 300 μm to about 500 μm, and a minimum width (e.g., “W2” of FIG. 3) of about, as examples, 1 μm to about 1,000 μm; about 1 μm to about 900 μm; about 1 μm to about 500 μm; about 1 μm to about 300 μm; about 1 μm to about 100 μm; or about 5 μm to about 50 μm. In an example, a diameter (or a width) (e.g., the width “W” of FIG. 1) in a well may uniformly decrease or increase in an upper end. In another example, a diameter (or a width) in a well may uniformly decrease or increase from an arbitrary position (i.e., a portion of a height). In a non-limited example, a bottom end (e.g., a bottom surface) of the well 210 may be flat, rounded, or conical.

In an example, referring to FIG. 2, a bottom end (e.g., a bottom surface) of the well 210 may be exposed to a lower layer (e.g., an electrode layer).

In an example, the well 210 may have a shape including at least one or a combination of a circle, an oval, a bead, a cylinder, a truncated cone, a polygonal pyramid, a polygonal prism, a tapered polygonal column, and a tapered and truncated cone, as only examples.

In an example, to control a floating culture and an adherent culture of cells (e.g., nerve cells) using a degradable well array layer, examples may provide an electrode system for a contact between electrodes and floating-cultured spheroids, for a control of an adhesion (e.g., an adhesion time or an adhesion strength), and for a formation and a control of a network between spheroids (e.g., a formation of a network and a control of a position of a network).

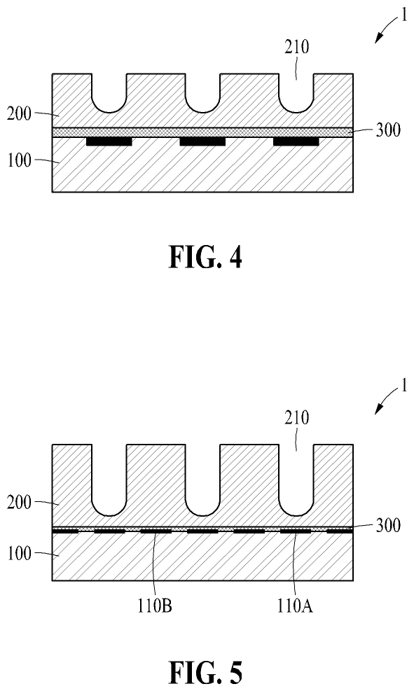

FIGS. 4 and 5 illustrate examples of a configuration of an electrode system 1.

Referring to FIGS. 4 and 5, the electrode system 1 may include an MEA layer 100, a well array layer 200 for a cell culture on the MEA layer 100, and an adhesion layer 300 between the MEA layer 100 and the well array layer 200.

The MEA layer 100 and the well array layer 200 have been described above with reference to FIGS. 1 to 3, and accordingly, further description thereof is not repeated herein.

In an example, the adhesion layer 300 may be exposed in a well (i.e., a bottom surface) of the well array layer 200.

In an example, the adhesion layer 300 may have a thickness of about 0.01 μm to about 1 μm; about 0.02 μm to about 1 μm; about 0.05 μm to about 1 μm; about 0.08 μm to about 1 μm; about 0.1 μm to about 1 μm; about 0.2 μm to about 0.8 μm; and about 0.4 μm to about 0.7 μm. In an example, the adhesion layer 300 may be formed on an electrode unit 110 of the MEA layer 100, to strengthen an adhesive force of the electrode unit 110, spheroids, and a network of the spheroids.

In an example, the adhesion layer 300 may be formed on a portion of the MEA layer 100, may be formed on the entire surface of the MEA layer 100, or may be formed within the MEA layer 100. In an example, the adhesion layer 300 may be disposed on an electrode layer (e.g., an electrode unit 110) corresponding to a well 210. In another example, the adhesion layer 300 may be disposed on all of the electrode units 110.

In an example, the adhesion layer 300 may include a material that provides an adhesion function for cell adhesion. In an example, a biopolymer adhesive may be applicable without a limitation. In an example, the biopolymer adhesive may be a biocompatible polymer, and may include at least one, or a combination of, polyacrylic acid, polyacrylamide, polyvinyl alcohol, polyhydroxy ethyl methacrylate, polyethylene glycol, polyvinylpyrrolidone, polyurethane, polydimethylsiloxane, polyvinyl chloride, styrene-ethylene-butylene-styrene (SEBS), gelatin, chitosan, alginate, polycaprolactone, polylactic acid, poly(lactic-co-glycolic acid), and polyhydroxyalkanoate (PHA). In an example, the biopolymer adhesive may further include a coupling agent or a cross-linking agent.

In an example, a surface of the adhesion layer 300 may be micro-textured. In an example, a micro-texture may be a nano-texture or a micro-texture. In an example, the micro-texture may include, for example, an embossed texture, a molded texture, a patterned texture, or a surface etched texture, as examples. In another example, the micro-texture may be a regular or irregular arrangement.

In an example, cells may be nerve cells, and spheroids may form a nerve cell modular network in which spheroids are connected to each other through axons during an adherent culture on an electrode layer.

In an example, the electrode system according to examples may be a device that may detect an electrical signal generated from a spheroid after the well array layer 200 is degraded. When spheroids are seated on an MEA after degradation of the wells 210, the spheroids may adhere to the MEA and may have surfaces modified to enable cell adhesion such that bundles of axons may spread out around the spheroids. In an example, an electrical stimulation, electroporation using a current injection, or an intracellular recording may be possible.

In an example, a cell recording device including the electrode system 1 may be provided. In an example, the cell recording device may be configured to culture cells in vitro or in vivo, collect cell information, stimulate cells, or electroporate cells. In an example, the cell recording device, which is a neural recording or stimulation device, may be an electrophysiological system-on-a-chip (SOC) to record a signal (e.g., an electrophysiological signal) of a nerve cell or stimulate a nerve cell. In an example, the cell recording device may be a cell image recording device.

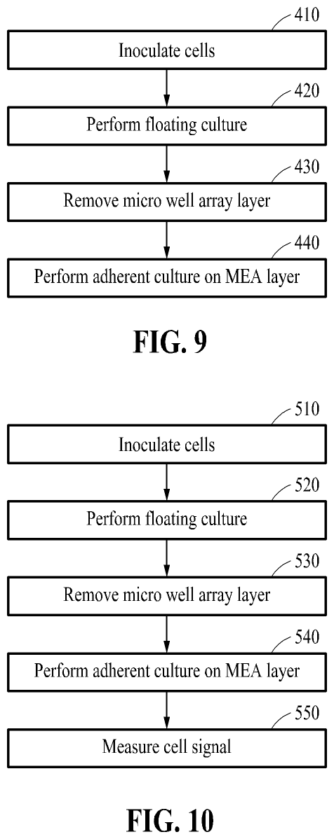

FIG. 9 is a flowchart illustrating an example method of forming a modular network of an electrode system 1. The operations in FIG. 9 may be performed in the sequence and manner as shown. However, the order of some operations may be changed, or some of the operations may be omitted, without departing from the spirit and scope. Additionally, some operations illustrated in FIG. 9 may be performed in parallel or simultaneously. In an example, the descriptions of FIGS. 1-8 may also be applicable to FIG. 9 and are incorporated herein by reference. Thus, the above description may not be repeated here for brevity purposes. The method of FIG. 9 may include operation 410 of inoculating cells into a micro well array layer (e.g., the well array layer 200 of FIG. 6 or 7), operation 420 of performing a floating culture, operation 430 of removing the micro well array layer, and operation 440 of performing an adherent culture on an MEA layer 100.

In an example, in the method of forming the modular network of the electrode system 1, an in-situ integration between multiple electrodes and the modular network may be achieved. In the in-situ integration, a 3D cell growth in the electrode system 1 and a formation of the modular network may be achieved.

In an example, the micro well array layer may include a well 210 including a degradable material, patterned on the MEA layer 100, and may be implemented based on a position in which a spheroid adheres.

In an example, in operation 420 of performing the floating culture, cells (e.g., nerve cells) may be cultured in vitro and grown may be achieved within a well 210. In an example, the floating culture may be performed to culture cells into spheroids or organoids.

In an example, in operation 430 of removing the micro well array layer, the well 210 may be self-degraded or may be degraded by an external factor (or stimulation), and cells (e.g., spheroids) cultured according to a pattern of the well array layer 200 may adhere onto the MEA layer 100.

In an example, in operation 440 of performing the adherent culture on the MEA layer 100, an adhesive force between cultured cells (e.g., spheroids) and electrode units 110 may be increased and a network between the cultured cells (e.g., spheroids) may be formed. In an example, a modular network may be formed by axons of nerve cells.

FIG. 10 is a flowchart illustrating an example method of recording cells using an electrode system. The operations in FIG. 10 may be performed in the sequence and manner as shown. However, the order of some operations may be changed, or some of the operations may be omitted, without departing from the spirit and scope. Additionally, some operations illustrated in FIG. 10 may be performed in parallel or simultaneously. In an example, the descriptions of FIGS. 1-9 may also be applicable to FIG. 10 and are incorporated herein by reference. Thus, the above description may not be repeated here for brevity purposes.

In an example, based on the method of FIG. 10, an in-situ integration between an MEA and a modular network may be utilized. In the in-situ integration, a 3D cell growth in the electrode system and a formation of the modular network may be achieved. The method of FIG. 10 may include operation 510 of inoculating cells into a micro well array layer (e.g., the well array layer 200 of FIGS. 6 and 7), operation 520 of performing a floating culture, operation 530 of removing the micro well array layer (e.g., the well array layer 200 of FIGS. 6 and 7), operation 540 of performing an adherent culture on an MEA layer (e.g., the MEA layer 100 of FIGS. 6 and 7), and operation 550 of measuring a cell signal.

According to an example, description of operations 510 to 540 of FIG. 10 may be the same as those of operations 410 to 440 of FIG. 9, and accordingly, further description thereof is not repeated herein.

In an example, operation 550 of measuring the cell signal may be performed during or after operation 540 of performing the adherent culture. In an example, in a process of forming a network between spheroids that adhere to the MEA layer, a cell signal (e.g., an electrical activity or an electrophysiological signal) may be measured. In an example, after a network between spheroids is formed, a cell signal (e.g., an electrical activity or an electrophysiological signal) may be measured.

In an example, examples may provide an electrode system (e.g., the electrode system 1 of FIGS. 1 to 5) that may construct a modular nerve cell network using an MEA in vitro and may detect a cell signal (e.g., an electrical activity or an electrophysiological signal) using the modular nerve cell network.

In an example, an electrode system (e.g., the electrode system 1 of FIGS. 1 to 5) may include a degradable micro well array layer in which micro-sized wells are patterned on an MEA, and a spheroid network patterned on a surface of the MEA may be formed using the degradable micro well array layer. In an example, the electrode system (e.g., the electrode system 1 of FIGS. 1 to 5) may arrange a modular nerve cell network on the surface of the MEA in vitro. In an example, a micro well array layer (e.g., the well array layer 200 of FIGS. 1 to 5) may be degradable by an external factor or an external environment and may control an adhesion and a 3D culture of cells by controlling a contact between cells and the surface of the MEA.

In an example, the electrode system (e.g., the electrode system 1 of FIGS. 1 to 5) may consecutively perform a floating culture and an adherent culture of cells (e.g., nerve cells) in implemented positions and may finely tune an arrangement pattern of spheroid networks.

Typically, to analyze operating principles of nerve cells, a dynamic measurement and analysis of a nerve cell activity may be desired. For the dynamic measurement and analysis, an MEA that electrically detects and records spikes generated from cells may be desired. In a multi-channel MEA, most electrodes may be arranged in a planar shape due to characteristics of a semiconductor device. Typically, to measure an activity of a nerve cell in vitro using an MEA, nerve cells extracted from a neural tissue should be separated in units of single neurons and cultured directly on the MEA. In this example, a connection between nerve cells on a two-dimensional (2D) plane may be formed as an all-to-all connection. Such an all-to-all connection network may be a structure that is not found in actual animal brains. A process of forming a 3D neuronal network to culture nerve cells in a floating state, instead of culturing nerve cells by adhering nerve cells to the bottom, may be used. To identify an electrical activity of the above 3D neuronal network, nerve cells may need to adhere again onto a 2D MEA or a multi-channel probe may need to be inserted. In a process of transferring a generated 3D neuronal network (e.g., a spheroid) to the MEA, a network between existing spheroids may be destroyed and a random network may be generated.

Thus, to solve the above-described problems, an electrode system (e.g., the electrode system 1 of FIG. 1, 2, 3, 4, or 5) that includes an MEA layer 100 and a well array layer 200 for a cell culture on the MEA layer 100, wherein the well array layer 200 includes a degradable material may be provided.

In an example, a floating culture and an adherent culture of cells (e.g., nerve cells) may be performed using the degradable well array layer 200, and an in-situ integration among spheroids, a network thereof, and an MEA may be implemented.

In an example, floating-cultured spheroids may be in contact with an area of an electrode unit 110 designated according to a pattern of the well array layer 200, and a network between the spheroids may be formed during the adherent culture. In other words, spheroids and a formation of a network thereof may be controlled by adjusting a degradation time or a degradation rate of the well array layer 200.

In an example, the well 210 may be micro-sized and may have a flat, rounded or conical bottom surface, as only examples. The well 210 may have a width that decreasing in a downwards direction, or may have a predetermined width.

In an example, the well 210 may have a width of about 1 μm to about 500 μm, as only examples.

In an example, one well 210 in the well array layer 200 may have a maximum width of about 100 μm to about 1,000 μm and a minimum width of about 1 μm to about 1,000 μm.

In an example, the well array layer 200 may include a degradable polymer, a degradable hydrogel, or both. Each of the degradable polymer and the degradable hydrogel may be included in a form of a fiber or a particle, or as a cross-linked network.

In an example, the well array layer 200 may be degraded and removed by a mechanical factor, a physical factor, or a chemical factor.

In an example, the well array layer 200 may be degraded by a factor including a temperature, a light, a change in pH, a vibration, an ultrasonic wave, or an acid.

In an example, the MEA layer 100 may include a plurality of electrode units 110. A portion (e.g., an electrode unit 110A of FIG. 3) of the plurality of electrode units 110 may be arranged in an area corresponding to the well 210, and the remaining electrode units (e.g., an electrode unit 110B of FIG. 3) may be arranged between wells 210. Each of the electrode units 110 may include a single electrode or a plurality of electrodes.

In an example, an electrode of the MEA layer 100 may be surface-modified or surface-textured with a chemical functional group.

In an example, the electrode system may further include an adhesion layer 300 that is formed between the well array layer 200 and the MEA layer 100.

In an example, the adhesion layer 300 may have a thickness of about 0.01 μm to about 1 μm, as only examples.

According to an example, cells may be floating-cultured into spheroids within the well 210. The spheroids may be in contact with an electrode layer corresponding to each well 210, and may be adherent-cultured, after the well 210 (or the well array layer 200) is removed. The spheroids may form a connection network with each other during the adherent culturing.

In an example, the cells may be nerve cells. The spheroids may form a connection network between the spheroids through a growth of axons during the adherent culturing.

In an example, an electrode system (e.g., the electrode system 1 of FIG. 1, 2, 3, 4, or 5) may measure cell information during or after an adherent culture of spheroids.

In an example, the electrode system (e.g., the electrode system 1 of FIG. 1, 2, 3, 4, or 5) may be configured to measure an electrical signal of a nerve cell.

In an example, a cell recording device including the electrode system (e.g., the electrode system 1 of FIG. 1, 2, 3, 4, or 5) may be provided.

In an example, the cell recording device may be configured to culture cells in vitro or in vivo, collect cell information, stimulate cells, or electroporate cells.

In an example, the cell recording device may be an electrophysiological SOC to record a signal of a nerve cell or stimulate a nerve cell.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. An electrode system, comprising:
a multi-electrode array (MEA) layer; and
a well array layer, disposed to support a cell culture, and disposed on the MEA layer,
wherein the well array layer comprises a degradable material.

2. The electrode system of claim 1, wherein:
a micro-sized well is disposed in the well array layer,
a bottom surface of the well has at least one of a flat shape, a rounded shape, and a conical shape, and
the well has a width that decreases in a downwards direction or has a predetermined width.

3. The electrode system of claim 1, wherein a well that is disposed in the well array layer has a width of 1 micrometer (μm) to 500 μm.

4. The electrode system of claim 1, wherein a well that is disposed in the well array layer has a maximum width of 100 μm to 1,000 μm, and a minimum width of 1 μm to about 1,000 μm.

5. The electrode system of claim 1, wherein:
the well array layer comprises a degradable polymer, a degradable hydrogel, or both, and
each of the degradable polymer and the degradable hydrogel is comprised in a form of one of a fiber and a particle, or is comprised as a cross-linked network.

6. The electrode system of claim 1 wherein the well array layer is degraded and removed by one of a mechanical factor, a physical factor, and a chemical factor.

7. The electrode system of claim 1, wherein the well array layer is degraded by a factor including at least one of a temperature, a light, a change in pH, a vibration, an ultrasonic wave, and an acid.

8. The electrode system of claim 1, wherein:
the MEA layer comprises a plurality of electrode units,
a first portion of the plurality of electrode units is arranged in an area corresponding to an area of wells of the well array layer, and a second portion of the plurality of electrode units are arranged between the wells of the well array layer, and
each of the electrode units comprises a single electrode or a plurality of electrodes.

9. The electrode system of claim 1, wherein an electrode of the MEA layer is surface-modified or surface-textured with a chemical functional group.

10. The electrode system of claim 1, further comprising:
an adhesion layer disposed between the well array layer and the MEA layer.

11. The electrode system of claim 10, wherein the adhesion layer has a thickness of 0.01 μm to 1 μm.

12. The electrode system of claim 1, wherein:
cells are floating-cultured into spheroids within a well that is disposed in the well array layer,
the spheroids are in contact with an electrode layer corresponding to the well and are adherent-cultured, after the well is removed, and
the spheroids form a connection network with each other during the adherent culturing.

13. The electrode system of claim 12, wherein:
the cells are nerve cells, and
the connection network is formed between the spheroids through a growth of axons during the adherent culturing.

14. The electrode system of claim 13, wherein the electrode system is configured to measure cell information during or after the adherent culturing of the spheroids.

15. The electrode system of claim 14, wherein the electrode system is configured to measure an electrical signal of the nerve cells.

16. A cell recording device, comprising:
an electrode system comprising:
a multi-electrode array (MEA) layer; and
a well array layer, disposed to support a cell culture, and disposed on the MEA layer,
wherein the well array layer comprises a degradable material.

17. The cell recording device of claim 16, wherein the cell recording device is configured to culture cells in vitro or in vivo, collect cell information, stimulate cells, or electroporate cells.

18. The cell recording device of claim 16, wherein the cell recording device is an electrophysiological system-on-a-chip (SOC) to record a signal of a nerve cell or stimulate a nerve cell.

19. The cell recording device of claim 16, wherein the degradable material comprises one of a degradable polymer and a degradable hydrogel.

20. The cell recording device of claim 16, wherein micro-sized wells are patterned on the MEA, and a spheroid network is patterned on a surface of the MEA.
